(19) United States
(12) Patent Application Publication
(10) Pub. No.: US 2012/0276245 A1
(43) Pub. Date: Nov. 1, 2012

(54) METHOD FOR IMPROVED FERMENTATION

(75) Inventors: Johannes Epeüs Theodoor Van Hylckama Vlieg, Marly le Roi (FR); Jeroen Hugenholtz, Parleiten-Geisenfeld (DE); Franciscus Adrianus Maria De Bok, Wageningen (NL); Sander Sieuwerts, Wageningen (NL)

(73) Assignee: STICHTING TOP INSTITUTE FOOD AND NUTRITION, Wageningen (NL)

(21) Appl. No.: 13/500,896

(22) PCT Filed: Oct. 7, 2010
(86) PCT No.: PCT/NL10/50660

§ 371 Date: Jul. 18, 2012

(30) Foreign Application Priority Data
Oct. 7, 2009 (EP) 09172434.4

(51) Int. Cl. C12N 1/38; A23P 1/00

(52) U.S. Cl. 426/7; 435/244

(57) ABSTRACT

The present invention discloses improved fermentation conditions for S. thermophilus and/or L. bulgaricus, allowing efficient preparation of fermented products based on monoculture of these strains. Such fermented products may be fermented food products or may be starter cultures for use in the preparation of fermented food products. The invention also describes the use of certain compounds for stimulating growth of S. thermophilus and/or L. bulgaricus.

### FIELD OF THE INVENTION

The present invention relates to the field of microbiology and food production using microbial fermentation in which the growth of a Streptococcus thermophilus strain in a fermentation medium is improved using a compound selected from the group consisting of pyruvic acid, folic acid and Tween-20, and the growth of a Lactobacillus bulgaricus strain in a medium is improved using a compound selected from the group consisting of sulfur-containing amino acids and branched-chain amino acids.

### BACKGROUND

Many food products are fermented by mixed cultures consisting of bacteria, yeasts or filamentous fungi. Fermented dairy products are typically produced with lactic acid bacteria (LAB), a prominent group of Gram-positive bacteria. Yogurt is bovine milk fermented by the LAB Streptococcus thermophilus and Lactobacillus delbrueckii subsp. bulgaricus (hereinafter also referred to as “L. bulgaricus” or “Lactobacillus bulgaricus”). During fermentation, both species contribute to the texture and the flavor of the product by (i) acidifying the medium leading to coagulation of the milk proteins, (ii) producing exopolysaccharides (EPS) and (iii) generating characteristic flavor compounds, such as acetaldehyde and diacetyl. S. thermophilus and L. bulgaricus stimulate each others' growth and acid production in a mixed milk culture, a process also referred to as protocooperation. This mutual stimulation is based on the exchange of growth enhancing metabolites. S. thermophilus provides L. bulgaricus with formic acid and folic acid and carbon dioxide, compounds that are all associated to purine biosynthesis either as precursors or cofactors. L. bulgaricus lacks pyruvate-formate lyase (PFL) and 2-amino-4-hydroxy-6-hydroxymethyldihydropteridine diphosphokinase, an essential gene in the biosynthetic pathway of folic acid. Other metabolic interactions exist at the level of nitrogen metabolism. Milk contains low levels of free amino acids (AA) and small peptides but milk proteins provide a rich source of AA that can be liberated through the action of extracellular proteolytic enzymes. Typically, the non-proteolytic S. thermophilus used in yogurt production profits from the proteolytic action of the membrane-resident protease prtB of L. bulgaricus. Similarly, L. bulgaricus was reported to be stimulated by long chain fatty acids (LCFA) such as oleic acid and lauric acid (Partanen et al. 2001. System. Appl. Microbiol. Vol. 24:500-506), but it remains to be established whether these are provided by S. thermophilus in mixed culture.

The metabolic interactions between the yoghurt bacteria have been elucidated mostly with classical microbiological approaches. More recently two postgenomic studies addressed the global response of S. thermophilus LMG18311 to growth in milk as a mono or mixed culture with L. bulgaricus ATCC11842 (Herve-Jimenez et al. 2009. Appl. Environ. Microbiol. Vol. 75, no. 7, p.2062-20′72; Herve-Jimenez et al. 2008. Proteomics, vol. 8:4273-4286). These studies revealed several additional metabolic responses to co-culture growth. The pathways for the biosynthesis of arginine and branched-chain AA (BCAA) were strongly upregulated in S. thermophilus in mixed culture. Also there was a pronounced response in iron metabolism. The authors showed that in response to H₂O₂ produced by L. bulgaricus, S. thermophilus shows multiple responses that may lead to lower intracellular iron concentrations. In this way S. thermophilus appears to minimize damage by reactive oxygen species (ROS) that are generated in the Fenton reaction.

The postgenomic analyses described above were only performed in S thermophilus. The present inventors aimed at analyzing the global regulatory responses to co-cultivation in milk in both Streptococcus thermophilus and Lactobacillus delbrueckii subsp. bulgaricus (herein also referred to as Lactobacillus bulgaricus or L. bulgaricus) simultaneously. The study was performed for several reasons. First of all, during yogurt production, cocultivation of both S. thermophilus and L. bulgaricus is required to obtain sufficient outgrowth of S. thermophilus to acidify the milk substrate. However, the presence of L. bulgaricus is disadvantageous for several reasons. L. bulgaricus is responsible for post-acidification during storage and distribution, rendering yogurt sour and less mild. Moreover, it produces off-flavours during yogurt production. As such, it would be advantageous to stimulate growth of S. thermophilus in the absence of L. bulgaricus, allowing yogurt-like production without post-acidification and off-flavour production. Secondly, the preparation of different types of yogurt starting cultures requires separate production of cultures of S. thermophilus and L. bulgaricus. Without the stimulatory effect of their protocooperation, growth of pure cultures of these bacteria is suboptimal. It would be advantageous to provide improved fermentation conditions for both S. thermophilus and L. bulgaricus for preparing individual starter cultures that may subsequently be used in the preparation of a fermented food product such as yogurt.

### SUMMARY OF THE INVENTION

The present inventors have identified compounds that stimulate growth of S. thermophilus and/or L. bulgaricus in monoculture, allowing these organisms to have enhanced growth in such monoculture or in mixed culture.

In a first aspect, the present invention relates to a method for preparing a fermented product, said method comprising the steps of:—Providing a fermentation medium comprising one or more first compounds, said first compound being selected from the group consisting of pyruvic acid, folic acid and Tween-20;—Adding a single acidifying strain to said fermentation medium, said acidifying strain being a Streptococcus thermophilus strain;—Optionally, adding one or more adjunct cultures to said fermentation medium; and—Allowing said fermentation medium to ferment to obtain a fermented product.

In an embodiment, said fermentation medium further comprises one or more second compounds, said second compound being selected from the group consisting of sulfur-containing amino acids, branched-chain amino acids, and formic acid.

Said fermented product may be a fermented food product or may be a Streptococcus thermophilus starter culture, such as for the preparation of yogurt.

In a second aspect, the present invention relates to the use of one or more first compounds, said first compound being selected from the group consisting of pyruvic acid, folic acid and Tween-20, in a fermentation medium for stimulating growth of Streptococcus thermophilus.

In an embodiment, one or more second compounds, said second compound being selected from the group consisting of sulfur-containing amino acids, branched-chain amino acids, and formic acid are further used for stimulating growth of S. thermophilus.

In a further aspect, the present invention provides for a method for preparing a fermented product, said method comprising the steps of:—Providing a fermentation medium comprising one or more third compounds, said third compound being selected from the group consisting of sulfur-containing amino acids and branched-chain amino acids;—Adding a single acidifying strain to said fermentation medium, said acidifying strain being a Lactobacillus delbrueckii subsp. bulgaricus strain;—Optionally, adding one or more adjunct cultures to said fermentation medium; and—Allowing said fermentation medium to ferment to obtain a fermented product.

In an embodiment, said fermentation medium further comprises one or more fourth compounds, said fourth compound being selected from the group consisting of formic acid, nucleobases such as purines, pyruvic acid, folic acid, Tween-20, and Tween-80.

The fermented product may be a fermented food product or may be a Lactobacillus delbrueckii subsp. bulgaricus starter culture, such as for the preparation of yogurt.

In another aspect, the present invention pertains to the use of one or more third compounds, said third compound being selected from the group consisting of sulfur-containing amino acids and branched-chain amino acids, for stimulating growth of Lactobacillus delbrueckii subsp. bulgaricus in a fermentation medium. One or more fourth compounds, said fourth compound being selected from the group consisting of formic acid, nucleobases such as purines, pyruvic acid, folic acid, Tween-20, and Tween-80, may further be used for stimulating growth of Lactobacillus delbrueckii subsp. bulgaricus.

### DETAILED DESCRIPTION OF THE INVENTION

S. thermophilus

In a first aspect, the present invention relates to a method for preparing a fermented product, said method comprising the steps of: a) Providing a fermentation medium comprising one or more first compounds, said first compound being selected from the group consisting of pyruvic acid, folic acid and Tween-20; b) Adding a single acidifying strain to said fermentation medium, said acidifying strain being a Streptococcus thermophilus strain; c) Optionally, adding one or more adjunct cultures to said fermentation medium; d) Allowing said fermentation medium to ferment to obtain a fermented product.

Surprisingly it has been found that pyruvic acid, folic acid and Tween-20 stimulate growth of S. thermophilus in monoculture, allowing improved growth thereof in monoculture in the absence of Lactobacillus bulgaricus. In such a manner, a yogurt-like product may be prepared without using Lactobacillus delbrueckii subsp. bulgaricus (herein also referred to as “Lactobacillus bulgaricus” or “L. bulgaricus”) which may cause post-acidification during storage and distribution of such yogurt and may produce off-flavours in said yogurt. Thus, in an advantageous embodiment, no L. bulgaricus is used in the fermentation using S. thermophilus.

A growth-enhancing or growth-stimulating amount of folate as referred to herein means about 0.01-500 ppm folate, preferably 0.1-250 ppm folate, preferably 0.5-50 ppm, more preferably 1-25 ppm, even more preferably 2.5-20 ppm.

A growth-enhancing or growth-stimulating amount of Tween-20 as referred to herein means about 1 μM to about 10 mM, preferably about 10 μM to about 5 mM, more preferably about 25 μM to about 2 mM, yet more preferably about 50 μM to about 1 mM, and even more preferably about 60 μM to about 0.5 mM.

A growth-enhancing or growth-stimulating amount of pyruvate as used herein refers to about 0.01 to about 100 mM, preferably about 0.1 to about 75 mM, more preferably about 0.5 to about 50 mM, yet more preferably about 1 to about 25 mM, and even more preferably about 1 to about 10 mM.

The method of the invention may comprise the steps of: i) providing a fermentation medium; ii) inoculating said fermentation medium with at least a S. thermophilus strain; iii) allowing fermentation to take place to obtain a fermentation product; and optionally iv) using all or part of the fermentation product for the preparation of a food product.

In an embodiment, one or more second compounds, said second compound being selected from the group consisting of sulfur-containing amino acids (methionine and/or cysteine), branched-chain amino acids (leucine, isoleucine and/or valine), and formic acid are further used for stimulating growth of S. thermophilus. Thus, a highly efficient fermentation medium may be composed allowing a higher growth rate and/or increased lactic acid production by S. thermophilus under fermentation conditions.

The first and/or second compounds may be added in a S. thermophilus growth-enhancing amount. It is within the routine skills of the skilled person to establish such S. thermophilus growth-enhancing amount of said first and/or second compounds. The skilled person may for example use the technique employed in Example 1 of the present invention, in which a certain amount of a compound is added and growth of S. thermophilus in the presence of said amount of the compound is compared to growth of S. thermophilus in the absence of said compound.

In an aspect, the present invention provides for the use of one or more first compound, said first compound being selected from the group consisting of pyruvic acid, folic acid and Tween-20, in a fermentation medium for stimulating growth of Streptococcus thermophilus. Advantageously, further one or more second compounds, said second compounds being selected from the group consisting of sulfur-containing amino acids, branched-chain amino acids, and formic acid, are used in said fermentation medium. The improved fermentation medium comprising said one or more first and/or second compounds may be used in monoculture of S. thermophilus, or maybe used in mixed culture of S. thermophilus and one or more further lactic acid bacteria. Preferably, said one or more further lactic acid bacteria do not comprise L. bulgaricus.

Lactobacillus delbrueckii subsp. bulgaricus

The present invention also provides for a method for preparing a fermented product, said method comprising the steps of:—Providing a fermentation medium comprising one or more third compounds, said third compound being selected from the group consisting of sulfur-containing amino acids and branched-chain amino acids;—Adding a single acidifying strain to said fermentation medium, said acidifying strain being a Lactobacillus delbrueckii subsp. bulgaricus strain;—Optionally, adding one or more adjunct cultures to said fermentation medium; and—Allowing said fermentation medium to ferment to obtain a fermented product.

In an embodiment, said fermentation medium further comprises one or more fourth compounds, said fourth compound being selected from the group consisting of formic acid, nucleobases such as purines, pyruvic acid, folic acid, Tween-20, and Tween-80.

Surprisingly it has been found that sulfur-containing amino acids and branched-chain amino acids stimulate growth of L. bulgaricus in monoculture, allowing growth thereof in monoculture in the absence of S. thermophilus. Such method is particularly advantageous in the preparation of L. bulgaricus starter cultures.

The method of the invention may comprise the steps of: i) providing a fermentation medium; ii) inoculating said fermentation medium with at least a L. bulgaricus strain; iii) allowing fermentation to take place to obtain a fermentation product; and optionally iv) using all or part of the fermentation product for the preparation of a food product.

In a further aspect, the invention is concerned with the use of one or more third compounds selected from the group consisting of sulfur-containing amino acids and branched-chain amino acids for stimulating growth of Lactobacillus delbrueckii subsp. bulgaricus in a fermentation medium. In an embodiment, one or more fourth compounds, said fourth compound being selected from the group consisting of formic acid, nucleobases such as purines, pyruvic acid, folic acid, Tween-20, and Tween-80, are further used in the fermentation medium. The improved fermentation medium for L. bulgaricus comprising said one or more third and/or fourth compounds may be used in monoculture of L bulgaricus, or may be used in mixed culture of L. bulgaricus and one or more further lactic acid bacteria.

The third and/or fourth compounds may be added in a L. bulgaricus growth-enhancing amount. It is within the routine skills of the skilled person to establish such L. bulgaricus growth-enhancing amount of said first and/or second compounds. The skilled person may for example use the technique presented in Example 1 of the present invention, in which a certain amount of a compound is added and growth of L. bulgaricus in the presence of said amount of the compound is compared to growth of L. bulgaricus in the absence of said compound.

Both S. thermophilus and L. bulgaricus

The fermentation medium may be any aqueous medium allowing its fermentation by S. thermophilus and/or L. delbrueckii subsp. bulgaricus. “Fermentation” or “fermentation culture” refers to growth cultures used for growth of bacteria which convert carbohydrates into alcohol and/or acids, usually (but not necessarily) under anaerobic conditions. “Fermentation medium” refers to the growth medium being used for setting up the fermentation culture, while “fermentation product” is generally used to refer to the fermented medium (i.e. during and/or after fermentation). However, both terms may be used interchangeably herein and the meaning will be clear from the context. The fermentation medium may be any fermentation medium comprising a sugar source, and a protein source. The sugar source may be any sugar that can be fermented by the S. thermophilus or L. bulgaricus strain used, and includes, without limitation, lactose, sucrose, dextrose, glucose, and the like. The protein source may be any protein source, including, but not limited to, milk proteins, vegetable proteins, including, without limitation, soy proteins, fish proteins, meat proteins, and the like. Particularly for the production of a fermented food product, it is preferred that the protein source is selected from milk proteins and vegetable proteins.

The fermentation product may be any fermentation product, but may also be a fermented food product, i.e. a liquid, semi-solid and/or solid food product (nutritional composition), suitable for human and/or animal consumption per se.

Thus, the fermentation product may be a fermented food product per se, such as yogurt or cheese, or the fermentation product may be used in the preparation of a food product. The term “food” or “food product” refers to liquid, semi-solid and/or solid food products (nutritional composition), suitable for human and/or animal consumption. The food or food product may be fermented per se (“a fermented food product”), e.g., yogurt, cheese, kefir, or the like, or may comprise a fermented food product or fermentation product prepared using the method of the present invention.

For example, the fermentation product may be used in other food products such as liquid foods (e.g. drinks, soups, yoghurts or yoghurt based drinks, milk shakes, soft drinks, fruit drinks, fermented dairy product, meal replacers, fermented fruit and/or juice products, etc.) or solid foods/feeds (meals, meal replacers, snacks such as candy bars, animal feed, fermented dairy products, fermented food or feed products, ice products, freeze dried food additives, cheeses, etc.) or semi-solid foods (deserts, etc.). The fermentation product may simply be added to, or used during the production process of such food products.

Alternatively, the fermentation product may be concentrated or diluted or pre-treated prior to being used to prepare a food composition. Pre-treatments include filtration and/or centrifugation, sterilization, freeze-drying, freezing, and the like. The fermentation product as such and/or the pre-treated fermentation product are in essence the primary products of the above method. These primary products may be used as such, e.g., in the case of fermented food products, or may be used as a food product ingredient, i.e. a suitable amount of primary product may be used as ingredient when making a final food product. The food composition according to the invention comprises or consists of a suitable amount of primary product (fermentation product, e.g. as such or pre-treated).

The fermentation product may be a starter culture, and may subsequently be used in the preparation of a food product, feed product, and the like. It has been found that the addition of one or more first and/or second compounds to a fermentation medium for S. thermophilus leads to efficient fast production of S. thermophilus comprising starter cultures, including an increased biomass production at the end of fermentation.

It has further been found that the addition of one or more third and/or fourth compounds to a fermentation medium for L. bulgaricus leads to efficient fast production of L. bulgaricus comprising starter cultures, including an increased biomass production at the end of fermentation. Prior to its use as starter culture, the fermentation product may be concentrated to provide a concentrated starter culture. The (concentrated) starter culture may be liquid, frozen or lyophilized. For use in the present invention, the (concentrated) starter culture may comprise the first and/or second compounds, or third and/or fourth compounds, referred to herein to provide an all-in-one package for the fermentation of a fermented food product. Alternatively, the (concentrated) starter culture and the first and/or second, or third and/or fourth, compounds may be added separately to the fermentation medium to provide a fermented food product.

The food product or fermentation product is preferably a fermented food product per se, including, but not limited to, a fermented dairy food product such as yogurt, cheese, kefir, buttermilk, sour cream, or soy yogurt, and the like. Such food product may further comprise common ingredients for the preparation of desserts, such as fruits, chocolate chips or cereals for example, but also sweetened products or liquid chocolates. The food product may further comprise common food ingredients such as emulsifiers, gelling agents, stabilizers, sweeteners, and the like. The person skilled in the art knows how to prepare a food product using the (fermented) food product of the present invention.

In a suitable embodiment, the fermented food product is a fermented dairy product. In an advantageous embodiment, the fermented food product is yogurt. For the preparation of yogurt of the present invention, a milk substrate may be fermented using S. thermopohilus as the single acidifying strain. Other bacteria, such as LAB, may be added, for example to provide the yogurt probiotic properties. S. thermophilus and L. bulgaricus are routinely used in yogurt and cheese preparation by fermenting a milk-type base fermentation medium comprising milk proteins, e.g., milk. It is also routinely used in the preparation of fermented soy food products, e.g., soy yogurt, using a soy-type base fermentation medium comprising 0.5-10% (w/w) soy protein, e.g., soy milk. S. thermophilus further requires a source of carbon and energy, such as a carbohydrate, e.g., a sugar such as lactose. Preferably, the milk-type base medium (also referred to as “milk substrate”) is natural or reconstituted milk, skimmed or otherwise, or milk-based media or media based on products of dairy origin.

The milk substrate or soy-type base medium may comprise items commonly used for the preparation of desserts or drinks, solid items such as fruits, chocolate chips or cereals for example, but also sweetened products or liquid chocolates.

Fermentation may take place using one or more adjunct starters, which includes yeasts such as those used in the preparation of Cheddar cheese, and bacteria. In an embodiment, the adjunct starters include bacterial strains, especially other lactic acid bacteria. “Lactic acid bacteria” (LAB) refers to bacteria, which produce lactic acid or another organic acid (such as propionic acid) as an end product of fermentation, such as, but not limited to, bacteria of the genus Lactobacillus, Streptococcus, Lactococcus, Oenococcus, Leuconostoc, Pediococcus, Carnobacterium, Propionibacterium, Enterococcus and Bifidobacterium.

Preferably, said one or more further bacterial strains are selected from Lactobacillus acidophilus, Lactobacillus casei and/or Bifidobacterium.

The fermentation medium comprising the one or more first and/or second compounds, or one or more third and/or fourth compounds, provides for an improved growth rate and/or increased lactic acid product for S. thermophilus and L. bulgaricus, respectively. A higher growth rate and/or increased lactic acid production may lead to enhanced food preservation and an improved texture of a fermented food product.

The term “sulfur-containing amino acids” refers to methionine and/or cysteine, whereas the term “branched-chain amino acids” or “BCAA” refers to leucine, isoleucine and/or valine.

The sulfur-containing amino acids and/or branched-chain amino acids may be provided in the form of free amino acids, or in the form of peptides comprising relatively large amounts, preferably at least 10%, 15%, 20%, 25%, 30%, 35%, 40%, 45%, 50%, on weight basis, of such sulfur-containing amino acids and/or branched-chain amino acids.

In this document, any and all S. thermophilus and L. delbrueckii subsp. bulgaricus strains are included, in particular those used for preparation of fermented (food) products.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition of the invention may comprise additional component(s) than the ones specifically identified, said additional component(s) not altering the unique characteristics of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

It will be clear that the above description and figure are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

### EXAMPLES

### Example 1

### Intervention Studies

Cultures of S. thermophilus, L. bulgaricus and a mixed culture were prepared in 0.8 volume reconstituted skim milk with 0.2 volume of a solution with compounds: Na-pyruvate (1.82 mM), Na-formate (1.47 mM), folic acid (1 mM), nucleobases (10 mg/L each) (all representing purine and pyrimidine metabolism), Tween-20 (105.9 μM) (as a supply of lauric acid), Tween-80 (110 mg/L) (as a supply of oleic acid). Here after these compounds are referred to as ‘interaction compounds’. The LCFAs oleic acid and lauric acid are poorly soluble and therefore we used Tween-20 and Tween-80. The effect of each of all interaction compounds on growth and acidification was tested in a single addition and a single omission strategy. Paired comparisons were made of a single compound versus nothing added (neg. control), and of all compounds minus one added versus all (pos. control). Acidification of quadruplicate cultures of 250 μL, was measured at 37° C. for 19 h in hydroplates (PreSens—Precision Sensing GmbH, Germany) where after CFU counts were determined using a rapid miniplating method (36). Significant differences in acidification were determined by comparing the maximal acidification rate using a two-tailed Students t-test (p=0.05). Similarly, significant differences were calculated between the final pH values and between the colony-forming units. A higher cell count, lower final pH, higher acidification rate and a shorter time to reach this rate are considered stimulatory effect of the intervention compared to the control.

Acidification by S. thermophilus was stimulated by the following compounds in decreasing order: formic acid, pyruvic acid, folic acid and Tween-20. L. bulgaricus acidification was stimulated the most by formic acid and nucleobases, whereas pyruvic acid, folic acid, Tween-20 and Tween-80 showed a small stimulatory effect.

Acidification of the mixed cultures was still stimulated by pyruvic acid, and formic acid, but in all cases stimulatory effects were less than with the mono-cultures.

### Example 2

Materials and Methods

Microarray Design

Microarrays were spotted on the Agilent 8x 15K platform (Agilent Technologies, Santa Clara, Calif., USA) with a custom probe design (AMADID 015342) comprising the sequences of both S. thermophilus CNRZ1066 (released by NCBI, genbank accession no. NC 006449) and L. bulgaricus ATCC BAA-365 (released by JGI, genbank accession no. NC 008529). The probes were designed with the objective to minimize cross-hybridization: the probes were species-specific, i.e. all probes were designed as 60-mers with a target score of 100% to the target gene, allowing no binding of cDNA that is 1 base different (mismatch) if the correct hybridization temperature (65° C.) and washing temperature (37° C.) are used. In total there are 5438 probes representing 1899 genes of S. thermophilus and 4028 spots representing 1709 genes of L. bulgaricus. Most genes are represented by 3 probes or more. Only 55 genes in S. thermophilus and 77 in L. bulgaricus are represented by one probe and only 5 genes of S. thermophilus and 31 genes of L. bulgaricus are lacking. The selectivity of strain specific gene detection was tested by a series of transcriptome profiling experiments of samples from MRS-grown mono-cultures of both strains. Comparative analysis of separate hybridizations and hybridization of a mixture of both samples showed that on average the probes showed 100-fold higher hybridization with RNA samples from the target strain. It was concluded that for a small number of genes strain specific gene expression analysis was not possible. These genes included rRNA genes (14 in S. thermophilus, 19 in L. bulgaricus), ribosomal proteins (4 and 12, respectively) and hypothetical proteins (8 and 2, respectively). They were excluded from further analysis.

RNA Isolation from Cultures Grown in Milk

The high protein content of milk and the polysaccharide production by the grown microorganisms make cell harvesting problematic. Furthermore, sampling and quenching need to be carried out rapidly in order to prevent the introduction of technical errors in a transcriptomics experiment. Several procedures have been developed to “clear” the milk to enable cell harvest by centrifugation without the contamination with milk solids. However, milk cleaning procedures are time consuming and require drastic changes in pH and the addition of large quantities of sodium citrate. We considered that this procedure is prone to lead to changes in the transcriptome. Therefore, we developed an alternative method for cell harvesting and RNA extraction from yoghurt cultures suitable for transcriptomic profiling. Yoghurt cultures were quenched in 3 volumes 60% glycerol of −40° C. leading to immediate arrest of cellular processes and kept at −20° C. for 0.5 h. Then pH was adjusted to 6.5-7.0 with 1 M NaOH and the medium was cleared with 4 mL 25% (w/v) Na₃Citrate per 100 mL at −20° C. for 0.5 h with gently mixing each 5 min. Cells were spinned down at −20° C. and 23000 G for 16 min and dissolved in a solution comprised off 50% (w/v) guanidinethiocyanate (Sigma), 0.5% (w/v) N-laurylsarcosine (Sigma) and 2.5% (v/v) of a 1 M sodium-citrate solution, adjusted to pH 7.0 with 0.1 M NaOH. After another centrifugation, the cells were resuspended in 500 μL 1xTE and applied to an RNA extraction tube containing 250 μL acidic phenol (Sigma), 250 μL chloroform (sigma), 30 μL NaAc (Merck) pH 5.2, 30 μL 10% SDS (Sigma) and 500 mg zirconium beads with 0.1 mm diameter (Biospec products Inc., OK, USA) which was immediately frozen in liquid nitrogen and kept at −80° C. until RNA extraction. For RNA isolation, a method was used that was already established for isolation from lactobacilli (Stevens et al., 2008. Improvement of Lactobacillus plantarum aerobic growth as directed by comprehensive transcriptome analysis. Appl Environ Microbiol 74:4776-4778). Briefly, cells were disrupted 3 times 45 s in a Fastprep (Qbiogene Inc., France) at 5.5 m/s separated by 1 min on ice. After centrifugation for 1 min at 20800 G, 500 μL of the aqueous phase was purified with 400 μL chloroform and a second centrifugation step. The aqueous phase was used for RNA isolation with a High Pure kit (Roche Diagnostics, Mannheim, Germany), which included 1 h of treatment with DNase I. RNA was stored at −80° C. Quantity and quality were checked using a ND-1000 photospectrometer (Nanodrop Technologies, Wilmington, Del., USA) and capillary electrophoresis on a RNA 6000 Nano LabChip® kit (Agilent Technologies, Santa Clara, Calif., USA) in a 2100 Bioanalyzer (Agilent). cDNA synthesis, labeling and hybridization

Five to seven μg of RNA was used for cDNA synthesis and labelling as described before (Stevens, 2008. Wageningen University, Wageningen, The Netherlands). For each array, 0.3 pg of cDNA labeled with Cyanine 3 and Cyanine 5 was hybridized. Hybridizations were performed with solutions and following the protocol delivered by

Agilent (version 5.5) for 8x 15K slides. Arrays were hybridized at 65° C. for 17 h. Hybridization schemes were designed that allowed duplicate comparisons between different stages within a fermentation experiment as well as and between mono and mixed cultures. Here after, the microarray slides were washed according to the manufacturer's instructions (buffer 1: room temperature, buffer 2: 30-37° C.) with the buffers supplied by Agilent. We found that washing at lower temperatures resulted in major cross-hybridization when hybridising S. thermophilus cDNA labelled with Cy5 and L. bulgaricus cDNA labelled with Cy3 simultaneously, but not when applying only one cDNA sample.

Array Analysis

Slides were scanned using an Agilent microarray scanner (G2565BA), Laser lights of wavelengths at 532 and 635 nm were used to excite Cyanine3 and Cyanine5 dye, respectively. Fluorescent images were captured as multi-image-tagged image file format and analyzed with Imagene software (Axon) (BioDiscovery, Marina del Rey, USA). The extent of hybridization was derived from a median value of pixel-by-pixel ratios. S. thermophilus and L. bulgaricus spots were normalised separately using Lowess (van Hijum, et al. 2008. BMC Bioinformatics 9:93.). Differential regulation was determined by false-discovery rate (FDR) from the Cyber-T p-values by means of multiple testing connection. Differential regulation was defined as a two-fold or higher differential expression with a FDR cut-off value of 0.05 or lower. Regulated genes were divided into functional classes as described by NCBI (S. thermophilus) and JGI (L. bulgaricus). Using Hierarchical clustering, principle component analysis and

MicroPrep, the quality of the different hybridizations was verified. Finally, results were visualized by plotting onto KEGG maps, Simpheny (Genomatica Inc., San Diego, Calif.), metabolic maps and Minomics.

Results

Transcriptome Analysis of Mono and Mixed Cultures

In order to identify genes that are differentially expressed in both species upon co-culture, transcriptome profiling was performed on mixed cultures and those were compared to mono-cultures at four different growth phases, i.e. the first exponential phase (3.5 h after starting the fermentation), transition phase (5.5 h), second exponential phase (8 h) and stationary phase (12 h). Similarly, we these four distinct growth phases were compared within a culture. Finally, transcriptome profiling was performed on cultures in early and mid second exponential phase mixed cultures supplemented with the interaction compounds formic acid and putrescine. These studies allowed analysis of global regulatory responses and the development of the interactions throughout the fermentation. DNA micro arrays were used that contained probes targeting strain-specific sequences ensuring minimal cross-hybridization for the genomes of both S. thermophilus CNRZ1066 and L. bulgaricus ATCC BAA-365. An RNA extraction method based on quenching by rapid freezing the culture and clarification by citrate was specifically designed for these experiments and proved to be crucial for the acquisition of high quality RNA samples from yoghurt cultures. Although we defined genes that were two-fold or more up or down-regulated with a FDR value of lower than 0.05 as significantly differentially expressed, also the more general effects were considered (e.g. all genes in a pathway are significantly upregulated by 1.5-fold).

Differential expression between mixed and mono-cultures was high in all four growth stages. The interactions affected S. thermophilus mainly in the second exponential phase (23% of all genes was more than 2-fold differentially expressed), which is in agreement with the observation that only at this growth phase S. thermophilus is profoundly stimulated by L. bulgaricus. The major functional groups affected included ‘Amino acid transport and metabolism’ (15-42% of the genes in the category), ‘Inorganic ion transport and metabolism’ (14-32%) and ‘Nucleotide transport and metabolism’ (10-47%). The presence of S. thermophilus stimulates L. bulgaricus growth already in the early stages of the fermentation, which is exemplified by the higher portion of differentially expressed genes in L. bulgaricus in the two early growth phases compared to S. thermophilus (24% versus 7% in the transition phase). A major part of the differential expression in both species could be attributed to the increased growth rate as is exemplified by the induction of primary metabolism including the genes involved in the production of important end products such as diacetyl, contributing to the typical yoghurt flavor. Indeed, this compound was present in larger quantities in mixed culture than in mono-culture. The major affected functional groups related to interactions included ‘Amino acid transport and metabolism’ (21-36% of the genes in the category), ‘Inorganic ion transport and metabolism’ (20-28%) and ‘Nucleotide transport and metabolism’ (18-44%).

Global Regulatory Responses Analysis of L. bulgaricus

In the L. bulgaricus mono-culture there was little difference in gene expression between the different growth phases except that from 8 h on (growth slows down and the culture enters stationary phase) many pathways were down-regulated, especially those associated with the biosynthesis of folic acid, purines, LCFA and AA and genes related directly related to growth such as those encoding ribosomal proteins and enzymes involved in cell wall biosynthesis. In the mixed culture there was a clear lower expression of genes associated with folic acid and purine biosynthesis, LCFA biosynthesis and sulfur AA metabolism in the transition phase compared to the first exponential phase. This may be due to the lower growth rate in the transition phase. In the second exponential phase, however, expression of purine and LCFA biosynthesis genes remained at a low level despite the higher growth rate compared to the transition phase. Moreover, LBUL_{—}0106, encoding 1-acyl-sn-glycerol-3-phosphate acyltransferase was expressed 13-fold higher, suggesting that this acyltransferase was loaded with LCFA harvested from the medium. In addition, genes involved in EPS and sulfur AA metabolism were higher expressed in the second exponential phase than in the transition phase.

Global Regulatory Responses in S. thermophilus

In the S. thermophilus mono-culture, the gene pflA (4.6-fold) for the production of formic acid and the pathway for purine biosynthesis were higher expressed in the transition phase compared to the first exponential phase despite the lower growth rate. Similarly, BCAA import and production genes were 2.9-3.0-fold higher expressed in the transition phase suggesting a shortage of these AA relatively early in the fermentation. Expression of genes for the production of other AA was in general lower in the transition phase compared to the first exponential phase. There was little difference in the second exponential phase compared to the transition phase except the up regulation of sulfur AA metabolism, as was also described by Herve-Jimenez et al. (supra) The trends in differential expression between the first exponential phase and the transition phase were comparable in S. thermophilus in mixed culture and the mono-culture, except for the fact that the higher expression of BCAA acquisition genes did not occur in the mixed culture. In the second exponential phase in mixed culture, purine biosynthesis genes were lower expressed than in the transition phase, but many pathways involved in AA acquisition were higher expressed, especially those for BCAA (2-3.1-fold) and sulfur AA (2.2-61.5-fold) suggesting an increased requirement for these AA. In the stationary phase, growth-related pathways were lower expressed. It is noteworthy that EPS biosynthesis genes of S. thermophilus were significantly higher expressed in the second exponential phase and stationary phase compared to the earlier growth phases in mixed culture, but not in mono-culture.

Purine Metabolism

found that the two genes for pyruvate formate lyase, pfl and pflA were higher expressed in mixed culture, especially in the first exponential phase (3.0 and 4.1-fold, respectively) compared to mono-cultures. Expression was down-regulated 3.8 and 5.7-fold when formic acid was supplied indicating that the physiological role of the enzyme was (in part) ensuring sufficient supply of formic acid. Expression of genes of the biosynthetic pathway for folic acid production was not affected, but expression of folic acid cycling genes (C1 pool) corresponded to the expression of genes for the production of purines. However, the incomplete folate biosynthetic pathway in L. bulgaricus was lower expressed, especially at the first two growth stages. Genes in the purine biosynthesis pathway in S. thermophilus were higher expressed in the mixed culture in the two earlier growth stages, but, in accordance to the study by Hervé-Jimenez et al. (supra), less expressed in the second exponential phase despite the higher growth rate. Similarly, purine metabolism in L. bulgaricus was lower expressed in mixed culture, especially after 5.5 h, potentially due to the lower growth rate in mixed culture at this phase. When formic acid was supplied, expression of genes involved in biosynthesis of purines and folic acid cycling was lower in the early (second) exponential phase but higher in the mid exponential phase in both species.

Amino Acid and Carbon Dioxide Metabolism

It is known that interactions occur at the level of nitrogen metabolism (proteolysis and carbon dioxide utilization). Nitrogen metabolism was poorly affected in L. bulgaricus with few exceptions. In co-culture we observed considerable higher expression levels of the prtB gene, LBUL_{—}1105, which was 8.9-fold higher expressed in the second exponential phase in co-culture. This can be explained by the fact that peptides generated upon casein hydrolysis by the protease are more rapidly consumed when S. thermophilus is also present. This demands higher protease activity to sustain growth of both bacteria. In addition, genes involved in the biosynthesis of the sulfur AA cysteine and methionine were highly upregulated in mixed culture, e.g. the gene that converts O-acetyl-L-serine into cysteine, LBUL_{—}1235, was expressed 23.1-fold higher in the mixed culture during the second exponential phase. This indicates that the proteolysis of casein does not allow the supply of sufficient cysteine for both organisms. Indeed, the cysteine content of casein is only 0.35. Moreover, the free methionine content of a milk culture is negligible and the free cysteine is rapidly consumed, i.e. cysteine does not accumulate in L. bulgaricus mono-culture and mixed culture, while several other AA do. In S. thermophilus, the higher peptide abundance due to the proteolysis executed by the protease that is produced by L. bulgaricus led to the upregulation of peptide import systems, such as the ABC transport system encoded by amiC, amiD, amiE and amiF1 (2.5-2.8-fold), and peptidolysis, as exemplified by the upregulation of the gene encoding peptidase PepN (2.4-fold) in the second exponential phase. In addition, genes encoding the biosynthesis of the three BCAA (2.0-fold) and uptake (1.0-1.3-fold) were slightly higher expressed in mixed culture. Similarly, in L. bulgaricus in mixed culture LBUL_{—}0431, encoding a branched-chain amino acid permease, was 2.3-fold higher expressed during the second exponential phase. That was anticipated since especially the S. thermophilus mono-culture and the mixed culture displayed a very low BCAA content, in particular of isoleucine. Similarly, in S. thermophilus there was a higher expression of pathways that convert serine into cysteine and methionine (1.5-1.9-fold). The pathways for de novo production of arginine out of glutamine and glutamate were upregulated in mixed culture. Glutamate is converted into ornithine mediated by four genes, argJ, argB, argC and argD, which were all 1.8-3.3-fold higher expressed in mixed culture at the second exponential phase.

In addition, carA, one of the genes responsible for the conversion of glutamine into carbamoyl phosphase, was 1.8 fold higher expressed. This all indicates that the urea cycle is running faster in S. thermophilus in the second exponential phase when grown in co-culture with L. bulgaricus. Moreover, cah, encoding carbonate dehydratase in S. thermophilus, was 3.8 to 15.8-fold higher expressed in mixed culture, in particular in the earlier growth phases. By liberating CO₂ from carbonate this enzyme may play a role in providing the CO₂ required for biosynthesis of aspartate, glutamate, arginine and nucleotides in both species. These results are in accordance with the results described by Hervé-Jimenez et al. (supra), who argued that BCAA and arginine metabolism in S. thermophilus were upregulated in presence of L. bulgaricus.

Fatty Acid Metabolism in L. bulgaricus

In the three later phases of fermentation, the genes encoding LCFA synthesis by L. bulgaricus were 3.3-9.6-fold lower expressed in mixed culture, while in the second exponential and the stationary phase LBUL_{—}0106 and LBUL_{—}1256 (both 1-acyl-sn-glycerol-3-phosphate acyltransferase) were 3.1 and 15-fold higher expressed in mixed culture, respectively. Therefore, it is likely that this acyltransferase is loaded with fatty acids from the medium in presence of S. thermophilus, e.g. liberated from milk fat by its lipolytic activity.

1. A method for preparing a fermented product, said method comprising:
Providing a fermentation medium comprising at least one first compound, said first compound being selected from the group consisting of folic acid, Tween-20, and pyruvic acid;
Adding a single acidifying strain to said fermentation medium, said acidifying strain comprising a Streptococcus thermophilus strain;
Optionally, adding one or more adjunct cultures to said fermentation medium;
Allowing said fermentation medium to ferment to obtain said fermented product.

2. The method according to claim 1, wherein said fermentation medium further comprises at least one second compound, said second compound being selected from the group consisting of sulfur-containing amino acids, branched-chain amino acids, and formic acid.

3. The method according to a claim 1, wherein said fermented product is a fermented food product.

4. The method according to claim 1, wherein said fermented product is a Streptococcus thermophilus starter culture, and is optionally a culture capable of being used for the preparation of yogurt.

5. A fermentation mediums comprising a first compound selected from the group consisting of folic acid, Tween-20, and pyruvic acid, wherein said first compound is used in said fermentation medium for stimulating growth of Streptococcus thermophilus.

6. A fermentation medium according to claim 5, further comprising at least one second compound, said second compound being selected from the group consisting of sulfur-containing amino acids, branched-chain amino acids, and formic acid.

7. A method for preparing a fermented product, said method comprising:
Providing a fermentation medium comprising at least one third compound, said third compound being selected from the group consisting of sulfur-containing amino acids and branched-chain amino acids;
Adding a single acidifying strain to said fermentation medium, said acidifying strain comprising a Lactobacillus delbrueckii subsp. bulgaricus strain;
Optionally, adding one or more adjunct cultures to said fermentation medium;
Allowing said fermentation medium to ferment to obtain said fermented product.

8. The method according to claim 7, wherein said fermentation medium further comprises at least one fourth compound, said fourth compound being selected from the group consisting of formic acid, nucleobases optionally comprising purines, pyruvic acid, folic acid, Tween-20, and Tween-80.

9. The method according to claim 7, wherein said fermented product is a fermented food product.

10. The method according to claim 7, wherein said fermented product is a Lactobacillus delbrueckii subsp. bulgaricus starter culture, optionally for preparation of yogurt.

11. A fermentation medium comprising a third compound selected from the group consisting of sulfur-containing amino acids and branched-chain amino acids for stimulating growth of Lactobacillus delbrueckii subsp. bulgaricus.

12. A fermentation medium according to claim 11, further comprising at least one fourth compound, said fourth compound being selected from the group consisting of formic acid, nucleobases optionally comprising purines, pyruvic acid, folic acid, Tween-20, and Tween 80.

13. The method according to claim 1, wherein at least one adjunct culture is added to said fermentation medium, said at least one adjunct culture being selected from the group consisting of bacteria of the genus Lactobacillus, Streptococcus, Lactobacillus, Streptococcus, Lactococcus, Oenococcus, Leuconostoc, Pediococcus, Carnobacterium, Propionibacterium, Enterococcus and Bifidobacterium.
